(19) United States
(12) Patent Application Publication
(10) Pub. No.: US 2026/0053983 A1
(43) Pub. Date: Feb. 26, 2026

(54) COATINGS FOR MEDICAL DEVICES AND RELATED METHODS

(71) Applicant: Boston Scientific Scimed, Inc., Maple Grove, MN (US)

(72) Inventors: Boxin TANG, St. Paul, MN (US); Michael BADGER, Princeton, MN (US); Victoria SZLAG, New Hope, MN (US); Christopher Michael HELTEMES, Big Lake, MN (US)

(73) Assignee: Boston Scientific Scimed, Inc., Maple Grove, MN (US)

(21) Appl. No.: 19/303,821

(22) Filed: Aug. 19, 2025

Related U.S. Application Data
(60) Provisional application No. 63/685,045, filed on Aug. 20, 2024.

(51) Int. Cl. A61L 27/34

(52) U.S. Cl. CPC A61L 27/34; A61L 2400/10; A61L 2420/02; A61L 2420/06

(57) ABSTRACT

Compositions such as medical devices that include a coating are described. The coating may include a polyvinylpyrrolidone polymer, a crosslinking agent, a photoinitiator, and a poloxamer. The crosslinking agent may include a polyfunctional ethylenically unsaturated monomer.

### CROSS-REFERENCE TO RELATED APPLICATION(S)

This application claims the benefit of priority to U.S. Provisional Application No. 63/685,045, filed on Aug. 20, 2024, which is incorporated by reference herein in its entirety.

### TECHNICAL FIELD

Aspects of the present disclosure relate generally to hydrophilic coatings for medical devices and related methods.

### BACKGROUND

Hydrophilic coatings may be used to impart lubricity to medical devices. For example, hydrophilic coatings may be useful for medical devices that are to be implanted or inserted into the body. During a medical procedure, hydrophilic coatings may reduce friction, e.g., between medical devices and/or between a medical device and tissue. A surface of a medical device may be treated with plasma prior to the application of a hydrophilic coating to, e.g., increase the wettability of the surface of the medical device. However, plasma treatment may increase costs and processing times.

### SUMMARY

The present disclosure includes, for example, a composition including a coating. The coating may include a polyvinylpyrrolidone polymer, a crosslinking agent, a photoinitiator, and a poloxamer. The crosslinking agent may include a polyfunctional ethylenically unsaturated monomer.

According to some aspects, the crosslinking agent may comprise neopentyl glycol diacrylate and/or the photoinitiator may comprise benzophenone. In some examples, the coating may comprise from about 0.01% by weight to about 0.15% by weight of the photoinitiator, relative to a total weight of the coating. In some examples, the coating may have a thickness less than or equal to 50 μm, e.g., a thickness ranging from about 5 μm to about 50 μm. In some examples, the poloxamer may have a molecular weight ranging from about 9,500 g/mol to about 15,000 g/mol, such as from about 11,500 g/mol to about 13,000 g/mol or from about 13,500 g/mol to about 15,000 g/mol. In some examples, the poloxamer may comprise poloxamer 338 or poloxamer 407. In some examples, the composition may comprise a polymer substrate and the coating may at least partially cover the polymer substrate. In some examples, the polymer substrate may comprise polyamide, polyurethane, polyethylene, or a combination thereof. The composition may be a medical device. The coating may be hydrophilic.

The present disclosure also includes methods of preparing the coatings described above and elsewhere herein. For example, the present disclosure includes methods of forming a coating on a polymer substrate. The method may comprise preparing a coating mixture by combining a polyvinylpyrrolidone polymer with a crosslinking agent, a poloxamer, a photoinitiator, and a solvent. In some examples, the crosslinking agent may comprise a polyfunctional ethylenically unsaturated monomer. In some examples, the solvent may comprise water and an alcohol. For example, the solvent may comprise water, an alcohol such as isopropanol, and an alkane such as heptane. The method may further include covering a surface of the polymer substrate with the coating mixture, and exposing the polymer substrate with the coating mixture to UV energy, thereby forming the coating on the polymer substrate.

According to some aspects, the polymer substrate may comprise polyamide, polyurethane, polyethylene, or a combination thereof. In some examples, the polymer substrate is untreated, e.g., the method does not include treating the polymer substrate with plasma before covering the surface of the polymer substrate with the coating mixture. In some examples, the poloxamer may have a molecular weight ranging from about 9,500 g/mol to about 15,000 g/mol, such as from about 11,500 g/mol to about 13,000 g/mol or from about 13,500 g/mol to about 15,000 g/mol.

The present disclosure also includes, for example, a composition comprising a polymer substrate at least partially covered by a coating, wherein the coating comprises a polyvinylpyrrolidone polymer, a crosslinking agent, a photoinitiator, and a poloxamer having a molecular weight ranging from about 9,500 g/mol to about 15,000 g/mol, such as from about 11,500 g/mol to about 13,000 g/mol or from about 13,500 g/mol to about 15,000 g/mol. In some examples, the crosslinking agent may comprise a polyfunctional ethylenically unsaturated monomer. In some examples, the coating may have a thickness less than or equal to 50 μm, such as a thickness ranging from about 5 μm to about 50 μm. In some examples, the crosslinking agent may comprise neopentyl glycol diacrylate. The composition may be a medical device. In some examples, the coating may be hydrophilic.

### BRIEF DESCRIPTION OF THE FIGURES

The accompanying drawings, which are incorporated in and constitute a part of this application, illustrate exemplary aspects of this disclosure and together with the description, serve to explain the principles of the present disclosure.

FIGS. 1A and 1B are plots of frictional force for coated substrates prepared with different concentrations of photoinitiator, as discussed in Example 1.

FIGS. 2A and 2B are plots of frictional force for various coated substrates, the coating comprising a poloxamer, as discussed in Example 2.

FIG. 3 is a plot of frictional force for various coated substrates, as discussed in Example 3.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ±5% of a specified amount or value. All ranges are understood to include endpoints, e.g., an amount ranging from 0.1 g to 10 g includes 0.1 g, 10 g, and all values between.

The present disclosure includes compositions comprising a coating. For example, the composition may comprise a substrate with a coating. The substrate may be or include a medical device, e.g., a medical device comprising a coating. The medical devices herein may be or include a catheter, a sheath, a dilator, a guidewire, a stent, a balloon, a patch, a sponge, or other medical device. The compositions herein may comprise a polymer substrate, e.g., a medical device comprising a polymer and/or having a polymeric surface. For example, the polymer substrate may comprise polyamide (e.g., nylon, polyether block amide (PEBA)), polyurethane, polyethylene (e.g., low-density polyethylene), or a combination thereof. The coatings herein may partially cover or completely cover the polymer substrate. The coatings herein may be hydrophilic to impart lubricity to the polymer substrate.

The present disclosure also includes methods for forming a coating on the polymer substrate (e.g., medical device) by applying a coating mixture comprising a polyvinylpyrrolidone polymer, a crosslinking agent, a poloxamer, a photoinitiator, and a solvent to the polymer substrate. The coating mixture(s) herein may be applied to a portion of or an entirety of the polymer substrate and then cured to form the coating on the polymer substrate. For example, the coating mixture(s) may be cured by exposure to ultraviolet (UV) energy, thereby forming the coating (e.g., a solid coating) on the polymer substrate. The coating may be at least partially crosslinked.

Typically, plasma treatment is used to form new oxygen-containing polar functional groups on a surface to, e.g., increase hydrophilicity and wettability of the surface. In some aspects, the methods herein do not include plasma treatment of the polymer substrate, e.g., the polymer substrate being untreated before applying the coating mixture. For example, one or more components of the coating mixture may facilitate spreading and/or adherence of the coating mixture to the polymer substrate, eliminating the need to treat the polymer substrate with plasma. The coating mixtures herein may reduce or prevent dewetting of the polymer substrate during the coating and/or UV curing processes.

According to some aspects of the present disclosure, an exemplary coating may comprise or consist essentially of a polyvinylpyrrolidone polymer, a crosslinking agent, a photoinitiator, and a poloxamer.

In some examples, the polyvinylpyrrolidone polymer may have an average molecular weight ranging from about 2,000 g/mol to about 2,000,000 g/mol, such as from about 2,000 g/mol to about 20,000 g/mol, from about 20,000 g/mol to about 50,000 g/mol, from about 50,000 g/mol to about 100,000 g/mol, from about 100,000 g/mol to about 500,000 g/mol, or from about 500,000 g/mol to about 2,000,000 g/mol. Non-limiting examples of PVP polymers suitable for the present disclosure include products under the names Kollidon® 12, Kollidon® 17, Kollidon® 25, Kollidon® 30, Kollidon® 90 and Luviskol® K90 by BASF Pharma, and PVP K-60 and PVP K-120 by Ashland.

In some examples, the crosslinking agent may comprise polyfunctional ethylenically unsaturated monomer(s) having two or more polymerizable ethylenically unsaturated groups. Exemplary crosslinking agents useful for the present disclosure include neopentyl glycol diacrylate (NPGDA), ethylene glycol diacrylate, di(ethylene glycol) diacrylate, tri (ethylene glycol) diacrylate, 1,4-butanediol diacrylate, and 1,6-hexanediol diacrylate.

In some examples, the photoinitiator may be used to facilitate crosslinking between the polyvinylpyrrolidone polymer and the polyfunctional ethylenically unsaturated monomer(s). Exemplary photoinitiators useful for the present disclosure include benzophenone, 2,2-dimethoxy-2-phenylacetophenone (Irgacure® 651), and 2-methyl-4′-(methylthio)-2-morpholinopropiophenone (Irgacure® 907).

In some examples, the poloxamer may be used to enhance the wetting properties of the coating, among other aspects. The poloxamer may have a molecular weight ranging from about 9,500 g/mol to about 15,000 g/mol, such as from about 10,000 g/mol to about 13,500 g/mol, from about 11,500 g/mol to about 13,000 g/mol, from about 9,500 g/mol to about 12,500 g/mol, or from about 13,500 g/mol to about 15,000 g/mol. Exemplary poloxamers useful for the present disclosure include, but are not limited to, poloxamer 338 and poloxamer 407.

An exemplary method of forming the coating on a polymer substrate will now be described. As mentioned above, the coating may be formed on the polymer substrate by applying a coating mixture to the polymer substrate. The method may include not treating the polymer substrate with plasma before covering a surface of the polymer substrate with the coating mixture.

The method may include preparing a coating mixture (e.g., a solution) by combining the polyvinylpyrrolidone polymer with the crosslinking agent, the photoinitiator, the poloxamer, and a solvent.

The solvent may be aqueous, e.g., comprising water, an alcohol, and an alkane. The solvent may comprise one or more molecules classified as class 3 solvents under the guidance of the International Council for Harmonisation of Technical Requirements for Pharmaceuticals for Human Use (ICH). Suitable alcohols include, but are not limited to, methanol, ethanol, and isopropanol. Alkanes suitable for the present disclosure may have a relatively high boiling point (e.g., greater than or equal to 95° C.) and a relatively low surface tension value, such as heptane. For example, the solvent may comprise water, isopropanol, and heptane. According to some aspects, the coating mixture may comprise from about 85% by weight to about 98% by weight of the solvent, e.g., from about 88% by weight to about 96% by weight, from about 90% by weight to about 95% by weight, from about 92% by weight to about 98% by weight, or from about 94% by weight to about 97% by weight, relative to the total weight of the coating mixture. For example, the coating mixture may comprise from about 10% by weight to about 15% by weight water, from about 75% by weight to about 85% by weight of an alcohol, and from about 2% by weight to about 8% by weight of an alkane. In another example, the coating mixture may comprise from about 8% by weight to about 12% by weight water, from about 80% by weight to about 85% by weight of an alcohol, and from about 3% by weight to about 6% by weight of an alkane.

The components of the coating mixture may be combined in any suitable order and thoroughly stirred or otherwise mixed until all solids are dissolved. For example, the polyvinylpyrrolidone polymer and poloxamer may be dissolved within an alcohol before, after, or at the same time the crosslinking agent and photoinitiator are added to the alcohol, followed by addition of water and an alkane. The coating mixture may be thoroughly stirred to provide for homogeneous mixing. In some examples, the coating mixture used to form the coating may comprise from about 2% by weight to about 7% by weight solids, and from about 93% by weight to about 98% by weight liquids.

In some examples, the coating mixture may comprise about 1.0% by weight to about 10.0% by weight of the polyvinylpyrrolidone polymer, relative to a total weight of the coating mixture, e.g., from about 2.0% by weight to about 6.0% by weight, from about 2.5% by weight to about 3.5% by weight, such as about 2.7% by weight of the polyvinylpyrrolidone polymer. Additionally or alternatively, the coating mixture may comprise about 0.2% by weight to about 1.0% by weight of the crosslinking agent, relative to a total weight of the coating mixture, e.g., from about 0.4% by weight to about 0.8% by weight, such as about 0.6% by weight of the crosslinking agent. Further, for example, the coating mixture may comprise about 0.01% by weight to about 0.15% by weight of the photoinitiator, relative to a total weight of the coating mixture, e.g., from about 0.01% by weight to about 0.1% by weight, from about 0.02% by weight to about 0.05% by weight, such as about 0.04% by weight of the photoinitiator. Additionally or alternatively, the coating mixture may comprise about 0.05% by weight to about 0.5% by weight of the poloxamer, relative to the total weight of the coating mixture, e.g., from about 0.1% by weight to about 0.3% by weight, from about 0.15% by weight to about 0.25% by weight, from about 0.18% by weight to about 0.22% by weight, such as about 0.2% by weight of the poloxamer.

The coating mixture may be applied to the polymer substrate by a suitable technique, such as dip-coating or spraying. The coating mixture once deposited on the polymer substrate may then be cured, e.g., by exposing the polymer substrate with the coating mixture to UV energy. For example, curing may be accomplished by incremental or continuous exposure to UV energy, optionally from multiple angles using spaced lamps and/or reflectors. For example, the coating mixture may be cured using a high intensity broad spectrum UV lamp.

The thickness of the coating may be selected based on the type of substrate (e.g., type of medical device) and desired use thereof. The viscosity of the coating mixture may be adjusted to provide for sufficient coverage of the substrate prior to curing in order to form the coating with the desired thickness. In some examples, the coating may have a thickness less than or equal to 50 μm, e.g., a thickness ranging from about 5 μm to about 50 μm, from about 10 μm to about 25 μm, from about 20 μm to about 35 μm, or from about 15 μm to about 40 μm.

Upon exposure to UV energy, the polyvinylpyrrolidone polymer may be crosslinked with the crosslinking agent and the photoinitiator in a polymer network, while the poloxamer is evenly distributed within the polymer network (not crosslinked). Upon exposure to UV energy, the crosslinking agent may undergo free radical polymerization and crosslinking, and the polyvinylpyrrolidone polymer may also undergo some crosslinking with the crosslinking agent.

Further, without being bound by theory, it is believed that the poloxamer may provide a synergistic effect with the solvent that inhibits or prevents dewetting of the polymer substrate. Without being bound by theory, it is believed that the poloxamer self-assembles into micelles in the coating mixture, reducing the surface tension of the coating mixture as compared to a mixture without the poloxamer. As alcohol of the solvent evaporates, surface tension of the coating mixture may increase, e.g., due to the presence of water and considering that water has a higher surface tension than alcohol and higher than alkanes. Accordingly, the presence of the poloxamer and the alkane may synergistically reduce surface tension (or minimize the increase in the surface tension of the coating mixture due to the evaporation of the alcohol), preventing or inhibiting dewetting of the polymer substrate.

### EXAMPLES

The following examples are intended to illustrate aspects of the present disclosure without, however, being limiting in nature. It is understood that the present disclosure encompasses additional embodiments consistent with the foregoing description and following examples. The present disclosure is not limited to the examples further described below and encompasses additional conditions without departing from the scope of the present disclosure.

### Example 1

Tests were conducted to assess different concentrations of photoinitiator on the lubricity and durability of coatings on different polymer substrates pretreated with plasma, in this case different medical devices. The coatings in this example did not include a poloxamer.

Coating mixtures A-F were prepared according to Table 1 below using benzophenone as the photoinitiator, polyvinylpyrrolidone (PVP), neopentyl glycol diacrylate (NPGDA) as the crosslinking agent, and a combination of isopropanol and water as the solvent. Coating mixture G was an external benchmark coating used for comparison.

TABLE 1

| Coating | Benzophenone | PVP | NPGDA | IPA | Water |
|---|---|---|---|---|---|
| Mixture | (wt. %) | (wt. %) | (wt. %) | (wt. %) | (wt. %) |
| A | — | 1.88 | 0.43 | 78.15 | 19.54 |
| B | 0.02 | 1.87 | 0.43 | 78.14 | 19.53 |
| C | 0.04 | 1.87 | 0.43 | 78.12 | 19.53 |
| D | 0.06 | 1.87 | 0.43 | 78.11 | 19.53 |
| E | 0.08 | 1.87 | 0.43 | 78.09 | 19.52 |
| F | 0.15 | 1.87 | 0.43 | 78.04 | 19.51 |

| | |
|---|---|
| G | External Coating |

Size 13 Fr sheaths of Pebax® and size 11 Fr dilators of polyurethane were used as the substrates and were treated with plasma before coating. Tests were conducted using the sheath substrates first followed by tests using the dilator substrates. The sheath substrates were dipped in respective coating mixtures A-G and the dilator substrates were dipped in respective coating mixtures A-D and F. The substrates were cured by UV exposure using an array of UV lamps at intensities between 1.5-2.9 mW/cm².

For lubricity and durability (L&D) testing, the substrates were cut into sample pieces. Each sample piece had a 10 cm minimum length. Distal and proximal sections of the substrates were tested using a Harland FTS 6000/7000 L&D tester with 1,000 g load cell force gauge and two 60D Silicone pads. Each test was set to run for 50 friction cycles and the clamp force was 800 g.

For each test, surfaces of the 60D Silicone pads were cleaned with 99% IPA wipes and rinsed with DI water, and then attached to the clamp of the tester. The clamp was then lowered into a container filled with DI water and submersed in the DI water without directly resting on the container. A sample piece was fastened to a sample holder and suspended from the 1,000 g load cell force gauge of the tester positioned above the container. The 1,000 g load cell force gauge was then lowered to place the sample piece between the 60D Silicone pads of the clamp and in the DI water. The sample piece was left in the DI water for 30 seconds before starting the test.

Each sample piece was tested and a frictional force required to move through the 60D Silicone pads was measured over the course of 50 friction cycles.

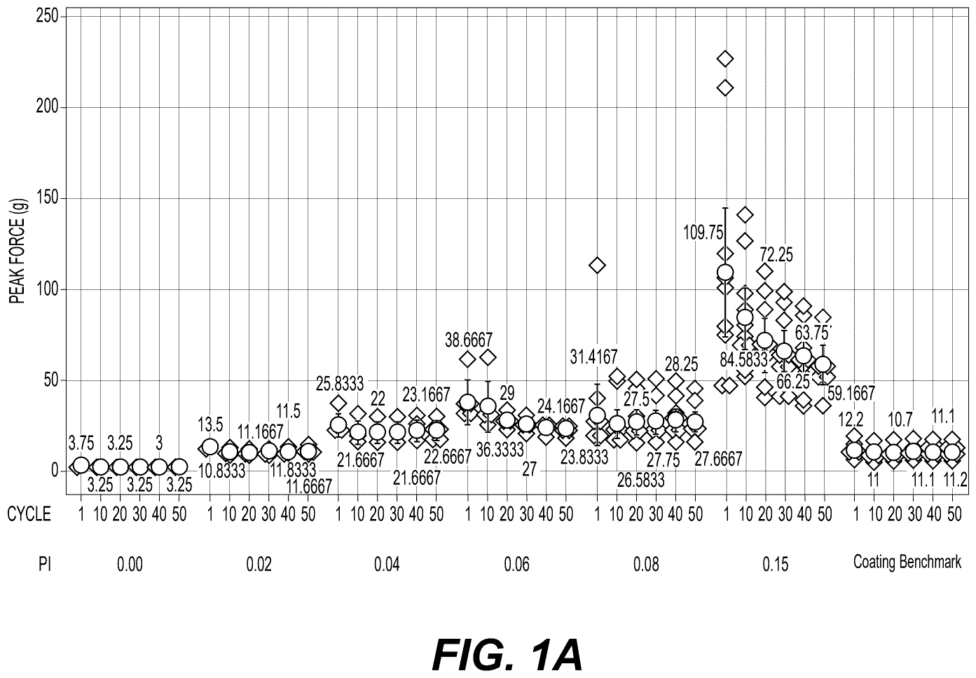

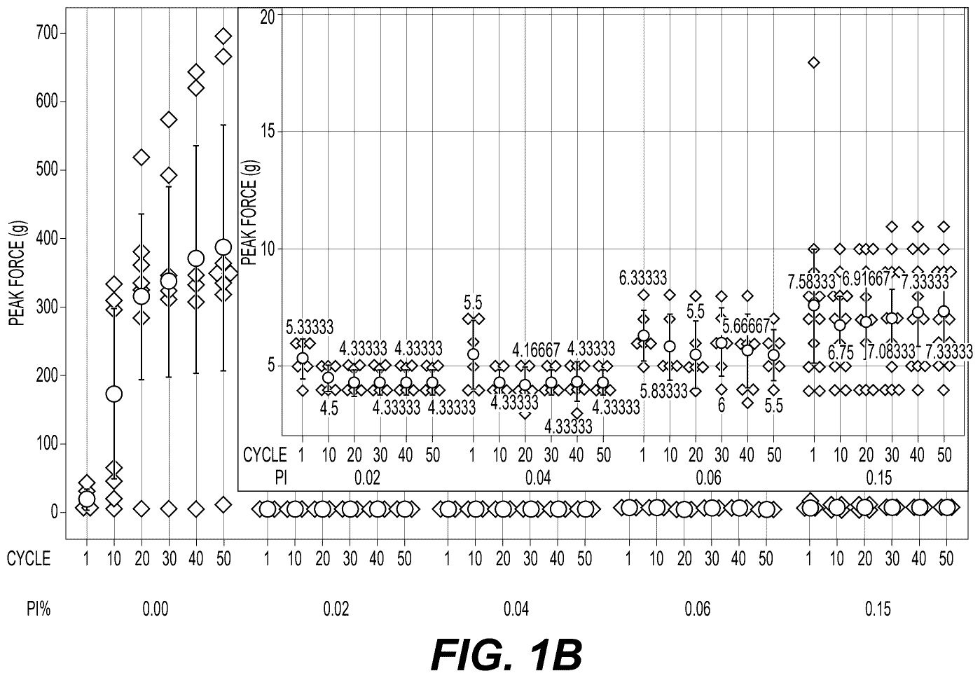

FIG. 1A is a plot of frictional force (g) versus number of friction cycles for the 13 Fr sheath sample pieces. FIG. 1B is a plot of frictional force (g) versus number of friction cycles for the 11 Fr dilator sample pieces. As shown in FIGS. 1A and 1B, coating mixture B (0.02 wt. % benzophenone) provided a coating with an average frictional force of less than 15.0 g over the course of 50 friction cycles in both the sheath and dilator sample pieces, indicating relatively high lubricity and durability. As shown in FIG. 1B, coating mixtures B-D and F provided improved lubricity and durability as compared to coating mixture A (no benzophenone).

### Example 2

Additional tests were conducted to assess the impact of a poloxamer and an alkane in the solvent used to prepare coatings. In this example, tests were conducted using different polymer substrates. The substrates were untreated, that is, not pre-treated with plasma.

In a first test, the substrates were dilators of low-density polyethylene. Coating mixtures H and I were prepared according to Table 2 below using benzophenone as the photoinitiator, PVP, NPGDA as the crosslinking agent, poloxamer 407 (Pluronic F127) as the poloxamer, and a combination of isopropanol, water, and heptane as the solvent.

TABLE 2

| | | | | | | | Poloxamer |
|---|---|---|---|---|---|---|---|
| Coating | Benzophenone | PVP | NPGDA | IPA | Water | Heptane | 407 |
| Mixture | (wt. %) | (wt. %) | (wt. %) | (wt. %) | (wt. %) | (wt. %) | (wt. %) |
| H | 0.04 | 1.87 | 0.43 | 78.12 | 19.53 | — | — |
| (control) | | | | | | | |
| I | 0.04 | 1.87 | 0.43 | 83.79 | 7.85 | 5.82 | 0.2 |

The substrates were dipped into coating mixtures H and I and cured using the same UV curing process described in Example 1. The substrates were then cut into sample pieces for lubricity and durability (L&D) testing. Each sample piece was tested using the same L&D testing procedure described in Example 1. Both distal and proximal sections of the substrates were tested.

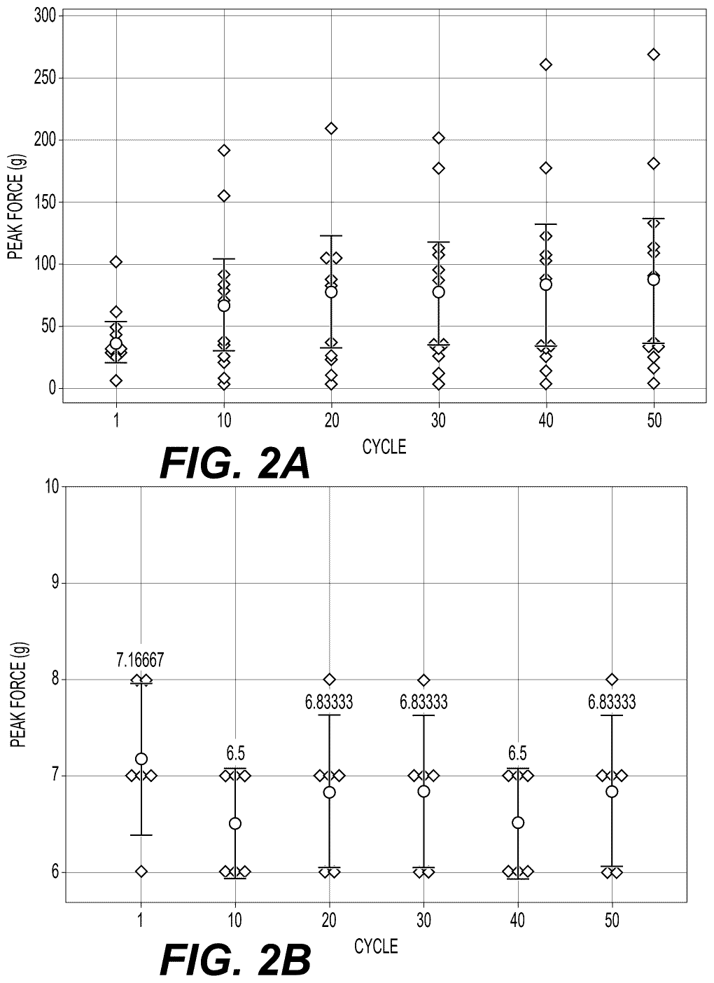

FIG. 2A is a plot of frictional force (g) versus number of friction cycles for coatings prepared with coating mixture H (control), showing the average frictional force increased to above 90.0 g over the course of 50 friction cycles indicating low lubricity and durability. FIG. 2B is a plot of frictional force (g) versus number of cycles for coatings prepared with coating mixture I, showing the average frictional force was less than 8.0 g across the course of 50 friction cycles and improved lubricity and durability as compared to coatings prepared with coating mixture H.

Proximal and distal sample pieces having coatings prepared with coating mixture I were imaged post-UV curing to assess coating uniformity. Those coatings were observed to be uniformly spread out on the surface of the sample pieces.

In a second test, size 13 Fr sheaths of Pebax® and size 11 Fr dilators of polyurethane were used as substrates. The substrates were dipped into coating mixture H (control) and cured using the same UV curing process described in Example 1. Proximal and distal sections of the substrates were imaged and dewetting of the substrates was observed.

### Example 3

Tests were conducted to assess the impact of sterilization on coatings prepared using coating mixture I in Example 2. Similar to Example 1, the substrates tested were size 13 Fr sheaths of Pebax® and size 11 Fr dilators of polyurethane. In this case, substrates were untreated, that is, not plasma pre-treated. The substrates were dipped into coating mixture I and then cured using the same UV curing process described in Example 1. A subset of the substrates was then sterilized using an ethylene oxide (EtO) sterilization process.

The sterilized substrates (test samples) and non-sterilized substrates (control samples) were then cut into sample pieces for lubricity and durability (L&D) testing. Each sample piece was tested using the same L&D testing procedure described in Example 1.

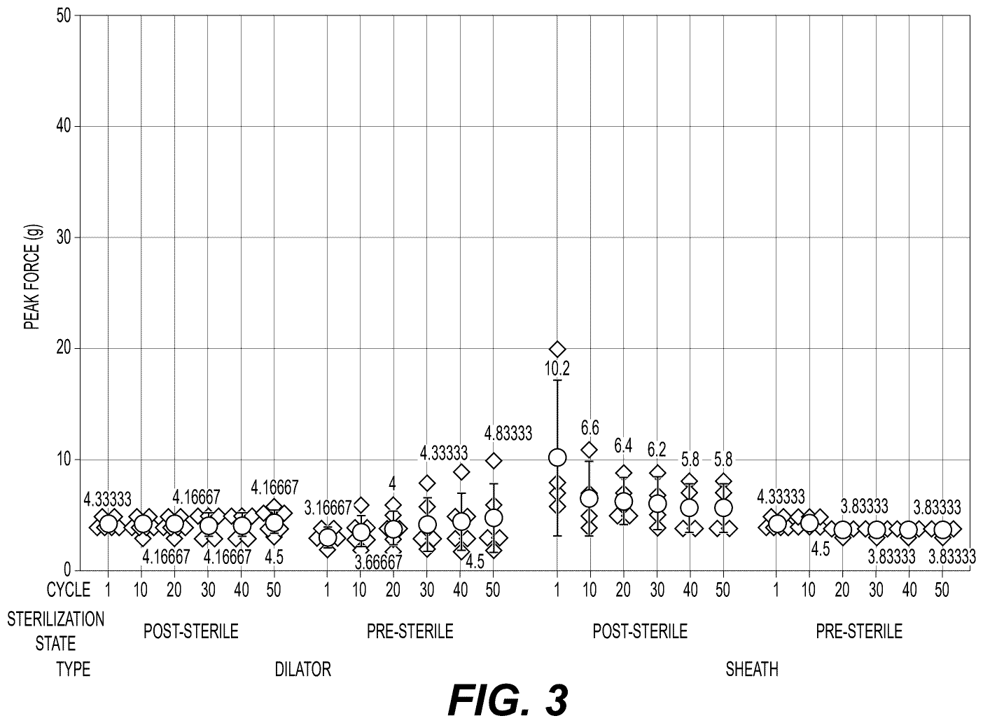

FIG. 3 is a plot of frictional force (g) versus number of friction cycles for the sterilized and non-sterilized sample pieces. As shown in FIG. 3, no significant difference in the average frictional force was observed, suggesting EtO sterilization did not significantly impact lubricity or durability of the coatings prepared with poloxamer and alkane in the solvent.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A composition comprising a coating, wherein the coating comprises:
a polyvinylpyrrolidone polymer;
a crosslinking agent comprising a polyfunctional ethylenically unsaturated monomer;
a photoinitiator; and
a poloxamer.

2. The composition of claim 1, wherein the crosslinking agent comprises neopentyl glycol diacrylate.

3. The composition of claim 1, wherein the photoinitiator comprises benzophenone.

4. The composition of claim 1, wherein the coating comprises from about 0.01% by weight to about 0.15% by weight of the photoinitiator, relative to a total weight of the coating.

5. The composition of claim 1, wherein the coating has a thickness less than or equal to 50 μm.

6. The composition of claim 1, wherein the poloxamer has a molecular weight ranging from about 9,500 g/mol to about 15,000 g/mol.

7. The composition of claim 1, wherein the poloxamer comprises poloxamer 407.

8. The composition of claim 1, wherein the composition comprises a polymer substrate, the coating at least partially covering the polymer substrate.

9. The composition of claim 8, wherein the polymer substrate comprises polyamide, polyurethane, polyethylene, or a combination thereof.

10. The composition of claim 1, wherein the composition is a medical device.

11. The composition of claim 1, wherein the coating is hydrophilic.

12. A method of forming a coating on a polymer substrate, the method comprising:
preparing a coating mixture by combining a polyvinylpyrrolidone polymer with a crosslinking agent, a poloxamer, a photoinitiator, and a solvent, wherein the crosslinking agent comprises a polyfunctional ethylenically unsaturated monomer, and wherein the solvent comprises water and an alcohol;
covering a surface of the polymer substrate with the coating mixture; and
exposing the polymer substrate with the coating mixture to UV energy, thereby forming the coating on the polymer substrate.

13. The method of claim 12, wherein the polymer substrate comprises polyamide, polyurethane, polyethylene, or a combination thereof.

14. The method of claim 12, wherein the method does not include treating the polymer substrate with plasma before covering the surface of the polymer substrate.

15. The method of claim 12, wherein the alcohol comprises isopropanol and the solvent further comprises an alkane.

16. The method of claim 12, wherein the poloxamer has a molecular weight ranging from about 9,500 g/mol to about 15,000 g/mol.

17. A composition comprising a polymer substrate at least partially covered by a coating, wherein the coating comprises:
a polyvinylpyrrolidone polymer;
a crosslinking agent comprising a polyfunctional ethylenically unsaturated monomer;
a photoinitiator; and
a poloxamer having a molecular weight ranging from about 9,500 g/mol to about 15,000 g/mol;
wherein the coating has a thickness less than or equal to 50 μm.

18. The composition of claim 17, wherein the crosslinking agent comprises neopentyl glycol diacrylate.

19. The composition of claim 17, wherein the composition is a medical device.

20. The composition of claim 17, wherein the coating is hydrophilic.
